(19) United States
(12) Patent Application Publication
(10) Pub. No.: US 2007/0021811 A1
(43) Pub. Date: Jan. 25, 2007

(54) Medical device including radiopaque polymer coated coil and method therefor

(75) Inventors: Peter J. D'Aquanni, Murrieta, CA (US); Aaron Baldwin, Temecula, CA (US); Vince Bavaro, Temecula, CA (US)

Correspondence Address: SCHWEGMAN, LUNDBERG, WOESSNER & KLUTH, P.A., P.O. BOX 2938, MINNEAPOLIS, MN, 55402, US

(73) Assignee: Cardiac Pacemakers, Inc.

(21) Appl. No.: 11/184,468

(22) Filed: Jul. 19, 2005

(51) Int. Cl. A61N 1/05

(52) U.S. Cl. 607/119

(57) ABSTRACT

An implantable medical device includes a radiopaque polymer coated coil.

### RELATED APPLICATIONS

This application is related to applications having Ser. No. 10/748,016, filed Dec. 29, 2003, Ser. No. 10/667,710 filed on Sep. 22, 2003, and Ser. No. 10/945,637, filed on Sep. 21, 2004, the entire contents of each are incorporated by reference herein.

### TECHNICAL FIELD

This relates generally to an implantable medical device having a radiopaque polymer coating thereon.

### BACKGROUND

Pacemaker leads represent the electrical link between the pulse generator and the heart tissue, which is to be excited and/or sensed. These pacemaker leads include one or more conductors that are connected to an electrode at an intermediate portion or distal end of a pacing lead.

To implant the lead within the patient, the lead is often fed intravenously toward the heart, for example, over a guidewire, or through a catheter. The lead may be implanted within or travel through complex or tortuous vasculature. The lead may also need to travel through vasculature having increasingly smaller diameters.

In order to visualize the lead, or guidewire, or catheter during implantation to facilitate travel through such difficult vasculature, many of the procedures are performed under fluoroscopy. Typically, radiopaque marker bands are placed along the device. However, the radiopaque markers are typically rigid relative to the device, and locally stiffen the device. Furthermore, the markers may provide inconsistent flexibility for the device. In addition, an implanting physician may be in need of information between the marker bands.

There is a need for medical devices with improved radiopaque qualities, without compromising other qualities of the devices.

### SUMMARY

A medical device is provided herein. The medical device includes a number of devices, such as, but not limited to, an intracorporeal intralumenal devices, guidewires, leads, stents, defibrillation leads, catheters, etc. The medical device includes at least one formed filament extending from a first end to a second end, where the filament is continuously coated with a radiopaque polymer material. Several options exist for the medical device. For instance, in one example option, the device further includes an adhesive disposed on an outer surface of the formed filament, and the adhesive bonds the formed filament with the radiopaque coating. In another example option, the coating has substantially the same or greater flexibility than the formed filament.

A method for forming the medical device is further provided herein. The formed medical device includes a number of devices, such as, but not limited to, guidewires, leads, stents, defibrillation leads, catheters, etc. The method includes continuously coating a flexible filament with a radiopaque polymer material along a length of the flexible filament, and forming the flexible filament into a medical device subsequent to coating the flexible filament.

Several options for the method exist. For instance, in one example option, the method further includes adhering the radiopaque material to the flexible filament, or spooling the flexible filament prior to forming the flexible filament into the medical device.

In another example method, a method for forming a medical device includes continuously coating a flexible radiopaque polymer directly on a flexible filament while forming the flexible filament, and forming the coated flexible filament into at least one of a medical device or component of a medical device subsequent to the coating. The formed medical device includes a number of devices, such as, but not limited to, guidewires, leads, stents, defibrillation leads, catheters, etc.

Options for the method include continuously coating and forming includes co-extruding the flexible filament with the flexible radiopaque polymer, or adhering the radiopaque polymer to the flexible filament, for instance during the continuous coating.

These and other embodiments, aspects, advantages, and features will be set forth in part in the description which follows, and in part will become apparent to those skilled in the art by reference to the following description and referenced drawings or by practice thereof. The aspects, advantages, and features are realized and attained by means of the instrumentalities, procedures, and combinations particularly pointed out in the appended claims and their equivalents.

### BRIEF DESCRIPTION OF THE DRAWINGS

In the drawing figures wherein like reference characters depict like parts throughout the same:

FIG. 1A illustrates a perspective of a medical device constructed in accordance with at least one embodiment.

FIG. 1B illustrates an end view of a medical device constructed in accordance with at least one embodiment.

FIG. 2 illustrates a side view of a guide wire constructed in accordance with at least one embodiment.

FIG. 3 illustrates a cross-sectional view of a catheter constructed in accordance with at least one embodiment.

FIG. 4 illustrates a side view of a defibrillation lead constructed in accordance with at least one embodiment.

FIG. 5 illustrates a block diagram of a method in accordance with at least one embodiment.

### DETAILED DESCRIPTION

In the following detailed description, reference is made to the accompanying drawings, which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the spirit and scope of the present invention. Therefore, the following detailed description is not to be taken in a limiting sense, and the scope is defined by the appended claims.

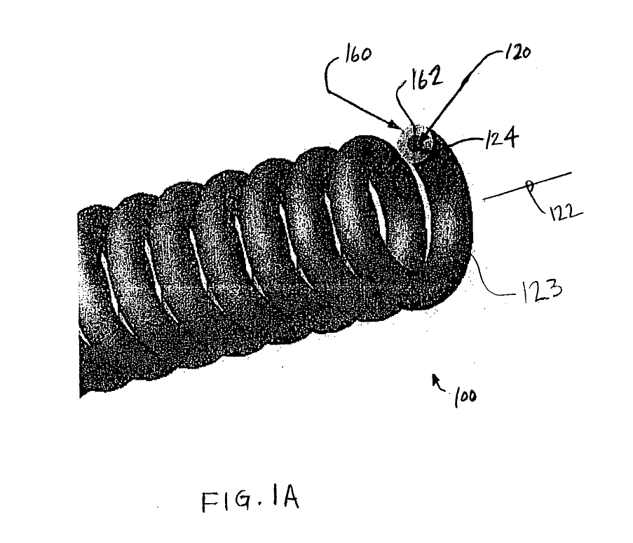

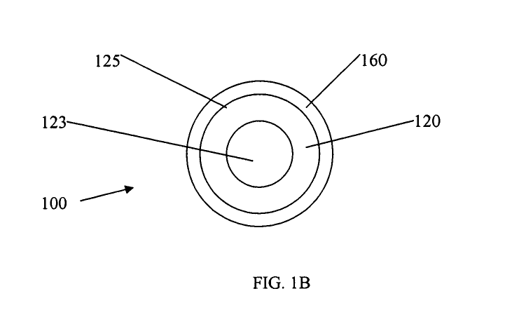

FIG. 1A illustrates one example of an implantable medical device 100, constructed in accordance with at least one embodiment. The medical device 100 includes a flexible filament 120, such as a coil. The medical device further includes a radiopaque polymer 160. The flexible, radiopaque polymer 160, in combination with the flexible filament 120, allows for the medical device to be easily viewed under fluoroscopy, without interfering with the performance or flexibility of the medical device.

The flexible filament 120 is formed from, in at least one option, a metallic material, such as a stainless steel, CoCr alloy, Ti alloy, or NiTi alloy. The flexible filament 120 is defined in part by a longitudinal axis 122 and a lumen 123 when it is disposed in a coiled arrangement. The flexible filament 120 is further defined in part by a filament outer surface 124. The radiopaque polymer 160 is disposed along the filament outer surface 124, for example, continuously along the flexible filament 120. In another example, as illustrated in FIG. 1B, the flexible filament 120 is coiled and has a coiled outer surface 125. The radiopaque polymer 160 is disposed along the coiled outer surface 125. For example, rather than placing discrete marker bands on the device, the radiopaque polymer 160 is continuously disposed along a length of the filament 120.

In another example, the filament 120 is formed into a coil and the coil is cut in to discrete lengths. The radiopaque polymer coated coil lengths are incorporated as radiopaque markers along the length of the medical device. For example, they can be disposed along an intermediate portion, or near the end, or at the tip of the device. In yet another example, the radiopaque polymer 160 extends substantially the full length of the filament 120 and/or the medical device 100. In a further option, adhesive 162 is disposed between the radiopaque polymer 160 and the flexible filament 120. In another example, adhesive is incorporated with the radiopaque polymer prior to the application of the radiopaque polymer material to the filament. Suitable examples of the adhesive include, but are not limited to, maleic acis anhydride.

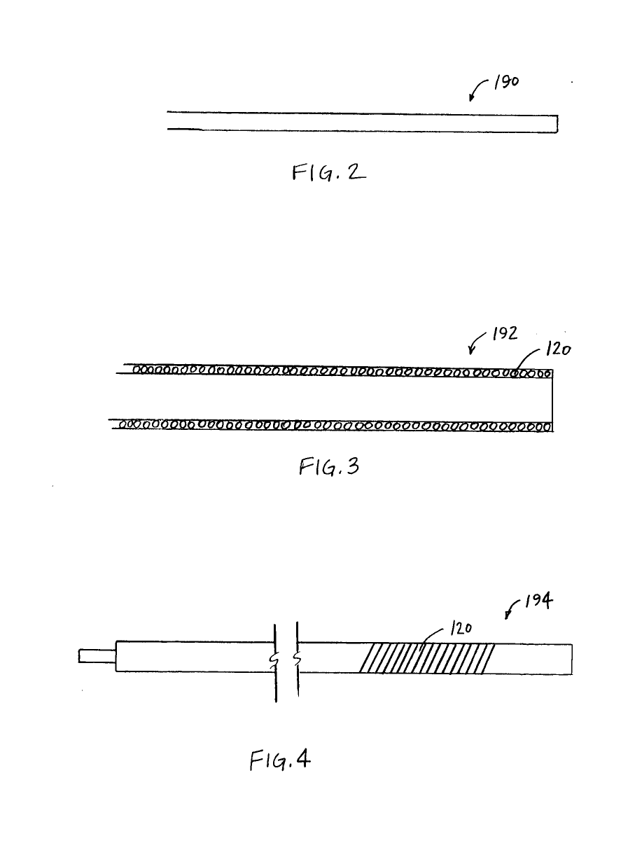

As mentioned above, the radiopaque polymer 160 is continuously coated, for example, continuously coated, on the outer surface of the flexible filament 120. In one example, the radiopaque polymer 160 is co-extruded with the flexible filament 120, as further described below. Optionally, the combination of the flexible filament 120 and the radiopaque polymer 160 are placed on a spool, for further processing. The spooled combination, can be formed into a variety of medical devices, such as a guide wire 190, as shown in FIG. 2, a catheter 192 as shown in FIG. 3, or a defibrillation lead 194 as shown in FIG. 4. The coated flexible filament can also be formed into a coil, and the coil is cut into discrete lengths. These coil lengths can be incorporated into the medical device, including, but not limited to, a guide wire, catheter, or defibrillation lead. For example, the coil can be cut into lengths such as, but not limited to, 1 mm-20 cm, and optionally bonded or connected with a non radiopaque coil.

Suitable materials for the radiopaque polymer 160 include, but are not limited to, a low durometer polymer in order to render the polymer sufficiently flexible so as not to impair the flexibility of the medical device 100. In another option the radiopaque polymer 160 has substantially the same or greater flexibility than the flexible filament 120. Examples of such polymers include, but are not limited to, polyamide copolymers like Pebax, polyetherurethanes like Pellethane, polyester copolymers like Hytrel, olefin derived copolymers, natural and synthetic rubbers like silicone and Santoprene, thermoplastic elastomers like Kraton and specialty polymers like EVA and ionomers, etc. as well as alloys thereof. Examples of radiographic materials include, but are not limited to, platinum, gold, iridium, palladium, rhenium, rhodium, tungsten, tantalum, silver and tin.

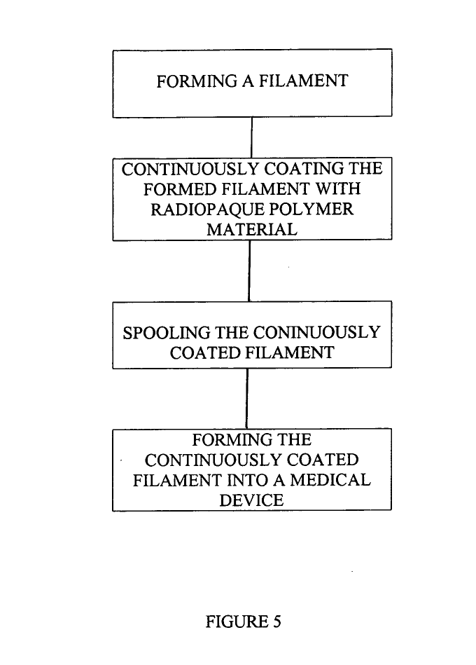

Manufacture of the radiopaque coated wire can be done in a number of manners. One example is illustrated in FIG. 5. In another example, the polymer resin is developed, which can optionally be first blended with a wetting agent. The blended polymer is fed into an extruder, for example, a twin screw extruder.

The materials are subjected to heat as they are conveyed through the extruder, causing the polymer to melt, thereby facilitating thorough homogenization of all of the ingredients. The radiopaque agent powder is subsequently introduced into the melt stream via a secondary feeder. The solid powder, molten polymer and additives are homogenized as they are conveyed downstream and discharged through a die as molten strands which are cooled in water and subsequently pelletized. The extrusion equipment employs two independent feeders as introduction of all components through a single primary feeder would require significantly higher machine torques and result in excessive screw and barrel wear. The powder feeder is operated in tandem with a sidefeeder device, which in turn conveys the powder through a sealed main barrel port directly into the melt stream.

After the radiopaque polymer material has been compounded, the medical device by an extrusion coating process. The flexible filament is fed through the extruder and the radiopaque polymer is continuously applied to the filament with the extruder. The coating adheres directly to the metal filament. The coated metal filament is coiled or spooled before, during, or after the extrusion process.

Advantageously, the continuous coating provides a way to effectively fluoroscopically visualize the various medical devices described above. The coating is placed in a coiled format directly on a metal wire, in some options, allowing for further fluoroscopic visualation.

It is to be understood that the above description is intended to be illustrative, and not restrictive. Although the use of the implantable device has been described for use with a lead in, for example, a cardiac stimulation system, the implantable device could as well be applied to other types of body stimulating systems. Many other embodiments will be apparent to those of skill in the art upon reviewing the above description. The scope should, therefore, be determined with reference to the appended claims, along with the full scope of equivalents to which such claims are entitled.

What is claimed is:

1. A medical device comprising:
at least one coiled metal filament extending from a first end to a second end; and
the at least one formed filament having a radiopaque coating thereon, wherein the metal filament is coated with a radiopaque polymer material.

2. The medical device as recited in claim 1, wherein the at least one coiled filament is a coiled wire having an outer coil diameter, and the radiopaque material is disposed on the outer coil diameter.

3. The medical device as recited in claim 1, wherein the filament has an outer filament diameter, and the radiopaque material is disposed on the outer filament diameter.

4. The medical device as recited in claim 1, wherein the radiopaque coating includes an adhesive therein disposed on an outer surface of the coiled filament, and the adhesive bonds the coiled filament with the radiopaque coating.

5. The medical device as recited in claim 1, wherein the continuous coating extends from the first end to the second end.

6. The medical device as recited in claim 1, wherein the medical device is a guidewire.

7. The medical device as recited in claim 1, wherein the medical device is a lead.

8. The medical device as recited in claim 7, wherein the medical device is a defibrillation lead.

9. A method comprising:
coating a metal flexible filament with a radiopaque polymer material along a length of the flexible filament including extruding the radiopaque material on to the flexible filament;
forming the metal flexible filament into a medical device subsequent to coating the flexible filament.

10. The method as recited in claim 9, further comprising adhering the radiopaque material to the flexible filament.

11. The method as recited in claim 9, further comprising spooling the flexible filament while extruding the flexible filament, and prior to forming the flexible filament into the medical device.

12. The method as recited in claim 9, further comprising forming the flexible filament into a guidewire.

13. The method as recited in claim 9, further comprising forming the flexible filament into a catheter.

14. The method as recited in claim 9, further comprising forming the flexible filament into a lead.

15. The method as recited in claim 14, further comprising forming the flexible filament into a defibrillation lead.

16. A method comprising:
continuously coating a flexible radiopaque polymer directly on a metal flexible filament while forming the flexible filament; and
forming the coated flexible filament into at least one of a medical device or component of a medical device subsequent to the coating.

17. The method of claim 16, wherein continuously coating and forming includes co-extruding the flexible filament with the flexible radiopaque polymer.

18. The method as recited in claim 16, further comprising adhering the radiopaque polymer to the flexible filament.

19. The method as recited in claim 18, wherein adhering the radiopaque polymer occurs during the continuous coating.

20. The method as recited in claim 16, further comprising forming the flexible filament into a guidewire.

21. The method as recited in claim 16, further comprising forming the flexible filament into a catheter.

22. The method as recited in claim 16, further comprising forming the flexible filament into a lead.

23. The method as recited in claim 22, further comprising forming the flexible filament into a defibrillation lead.
